(19) United States
(12) Patent Application Publication
(10) Pub. No.: US 2016/0089188 A1
(43) Pub. Date: Mar. 31, 2016

(54) SPINAL IMPLANT SYSTEM AND METHOD

(71) Applicant: Warsaw Orthopedic, Inc., Warsaw, IN (US)

(72) Inventors: Larry T. McBride, JR., Memphis, TN (US); Nicholas Benson, Cordova, TN (US)

(21) Appl. No.: 14/496,927

(22) Filed: Sep. 25, 2014

(51) Int. Cl. A61B 17/70

(52) U.S. Cl. CPC A61B 17/7076

(57) ABSTRACT

A method comprises the steps of: providing a first surgical instrument comprising a surface having a mating element; connecting the first surgical instrument with a fastener, connecting the fastener with tissue; engaging the mating element with a mating element of at least one of a plurality of alternate second surgical instruments interchangeable with the first surgical instrument; and treating the spine with the first surgical instrument and the at least one second surgical instrument. Instruments and implants are disclosed.

### TECHNICAL FIELD

The present disclosure generally relates to medical devices for the treatment of musculoskeletal disorders, and more particularly to a surgical implant system and a method for treating a spine.

### BACKGROUND

Spinal pathologies and disorders such as scoliosis and other curvature abnormalities, kyphosis, degenerative disc disease, disc hemiation, osteoporosis, spondylolisthesis, stenosis, tumor and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, correction, discectomy, laminectomy and implantable prosthetics. As part of these surgical treatments, spinal constructs, such as, bone fasteners, spinal rods and interbody devices can be used to provide stability to a treated region. During surgical treatment, surgical instruments can deliver components of the spinal constructs to the surgical site for fixation with bone to immobilize a joint. For example, spinal rods may be attached via the fasteners to the exterior of two or more vertebral members. The rods can redirect stress away from a damaged or defective region while healing takes place to restore proper alignment and generally support the vertebral members. This disclosure describes an improvement over these prior art technologies.

### SUMMARY

In one embodiment, a system and method for treating a spine are provided. The method comprises the steps of: providing a first surgical instrument comprising a surface having a mating element; connecting the first surgical instrument with a fastener; connecting the fastener with tissue; engaging the mating element with a mating element of at least one of a plurality of alternate second surgical instruments interchangeable with the first surgical instrument; and treating the spine with the first surgical instrument and the at least one second surgical instrument. In some embodiments, instruments and implants are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure;

FIG. 1A is a perspective view of components of the system shown in FIG. 1;

FIG. 1B is a perspective view of components of one embodiment of a system in accordance with the principles of the present disclosure;

FIG. 1C is a break away cross section view of the components shown in FIG. 1B;

FIG. 1D is a break away cross section view of the components shown in FIG. 1B;

FIG. 1E is a perspective view of components of one embodiment of a system in accordance with the principles of the present disclosure;

FIG. 1F is a perspective view of the components shown in FIG. 1E;

FIG. 2 is a lateral view of one embodiment of a system in accordance with the principles of the present disclosure disposed with a body;

FIG. 2A is a perspective view of a component shown in FIG. 2;

FIG. 2B is a lateral view of components of the system shown in FIG. 2;

FIG. 3 is a perspective view of components of one embodiment of a system in accordance with the principles of the present disclosure;

FIG. 3A is a perspective view of components shown in FIG. 3 with parts separated;

FIG. 4 is a perspective view of components of one embodiment of a system in accordance with the principles of the present disclosure;

FIG. 5 is a perspective view of components of one embodiment of a system in accordance with the principles of the present disclosure;

FIG. 6 is a perspective view of components of one embodiment of a system in accordance with the principles of the present disclosure disposed with vertebrae;

FIG. 7 is a side view of components of one embodiment of a system in accordance with the principles of the present disclosure;

FIG. 8 is a perspective view of components of one embodiment of a system in accordance with the principles of the present disclosure;

FIG. 8A is a perspective view of components of the system shown in FIG. 8;

FIG. 9 is a side view of components of one embodiment of a system in accordance with the principles of the present disclosure;

FIG. 10 is side view of components of one embodiment of a system in accordance with the principles of the present disclosure;

FIG. 11 is a perspective view of components of one embodiment of a system in accordance with the principles of the present disclosure disposed with vertebrae;

FIG. 12 is a side view of components of one embodiment of a system in accordance with the principles of the present disclosure disposed with vertebrae;

FIG. 13 is a side cross section view of components of one embodiment of a system in accordance with the principles of the present disclosure;

FIG. 13A is a break away cross section view of the components shown in FIG. 13;

FIG. 14 is a perspective view of components of one embodiment of a system in accordance with the principles of the present disclosure; and

FIG. 15 is a perspective view of components of one embodiment of a system in accordance with the principles of the present disclosure disposed with vertebrae.

### DETAILED DESCRIPTION

The exemplary embodiments of the surgical system and related methods of use disclosed are discussed in terms of medical devices for the treatment of musculoskeletal disorders and more particularly, in terms of a surgical implant system for implant delivery to a surgical site and a method for treating a spine. In some embodiments, the systems and methods of the present disclosure are employed with a spinal joint fixation, for example, with a cervical, thoracic, lumbar and/or sacral region of a spine.

In one embodiment, the surgical implant system can include a bone fastener that allows capture and retention under tension and compression. In some embodiments, compression may be applied to the bone fastener in a cephalad/caudal direction or a lateral direction. In some embodiments, the tension may be applied to the bone fastener through a surgical instrument or an implant support, such as, for example, an extender and compression may be applied through a sleeve of the extender. In one embodiment, the surgical system of the present disclosure includes an extender that employs relative axial translation of its component parts to dispose its components in a lock, load and/or an eject position to capture an implant such as a head of a bone screw.

In some embodiments, the surgical implant system may be employed with a method for treating a spine. In some embodiments, the method comprises a step of providing a first surgical instrument comprising a surface having a mating element. In some embodiments, the method comprises a step of connecting the first surgical instrument with a fastener. In some embodiments, the method comprises a step of connecting the fastener with tissue. In some embodiments, the method comprises a step of engaging the mating element with a mating element of at least one of a plurality of alternate second surgical instruments interchangeable with the first surgical instrument. In some embodiments, the method comprises a step of treating the spine with the first surgical instrument and the at least one second surgical instrument.

In some embodiments, the method comprises a step of providing at least one extender comprising a surface having a mating element. In some embodiments, the method comprises a step of connecting the at least one extender with a bone screw. In some embodiments, the method comprises a step of connecting the bone screw with a vertebral level. In some embodiments, the method comprises a step of engaging the mating element with a mating element of at least one of a plurality of alternate second surgical instruments interchangeable with the at least one extender. In some embodiments, the method comprises a step of treating the spine with the first surgical instrument, the at least one second surgical instrument and a complementary surgical instrument.

In some embodiments, the surgical implant system comprises a fastener, a first surgical instrument comprising a surface having a mating element and a capture element engageable with the fastener and at least one of a plurality of alternate second surgical instruments. The at least one second surgical instrument has a mating element engageable with the mating element of the first surgical instrument such that the at least one second surgical instrument is interchangeable with the first surgical instrument in a configuration for use with one or a plurality of different surgical procedures for treating a spine.

In some embodiments, the system may include instruments that are connected or attached to an extender(s) connected with a lateral translation handle or derotation instruments. In some embodiments, the system may have an extender with a quick release mechanism to allow the extender to slide into engagement with an implant. In some embodiments, the system can include an extender having features that prevent an implant from rotating.

In one embodiment, the surgical system of the present disclosure includes an outer assembly having clearance for mating with surgical instruments, such as, for example, a rod reducer. In some embodiments, the surgical system includes an extender that is tapered to create a smooth transition from an implant to the assemblies. In one embodiment, the extender includes openings that facilitate passage of a spinal rod through a window of the extender and prevents passage of the rod through the window adjacent other portions of the extender.

In one embodiment, the surgical system comprises surgical instruments that include image guided technologies, such as, for example, surgical navigation components employing emitters and sensors, which may be employed to track introduction and/or delivery of the components of the surgical system including the surgical instruments to a surgical site.

In one embodiment, one or all of the components of the system are disposable, peel-pack, pre-packed sterile devices used with an implant. One or all of the components of the system may be reusable. The system may be configured as a kit with multiple sized and configured components.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context dearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, muscle, ligaments, tendons, cartilage and/or bone unless specifically referred to otherwise.

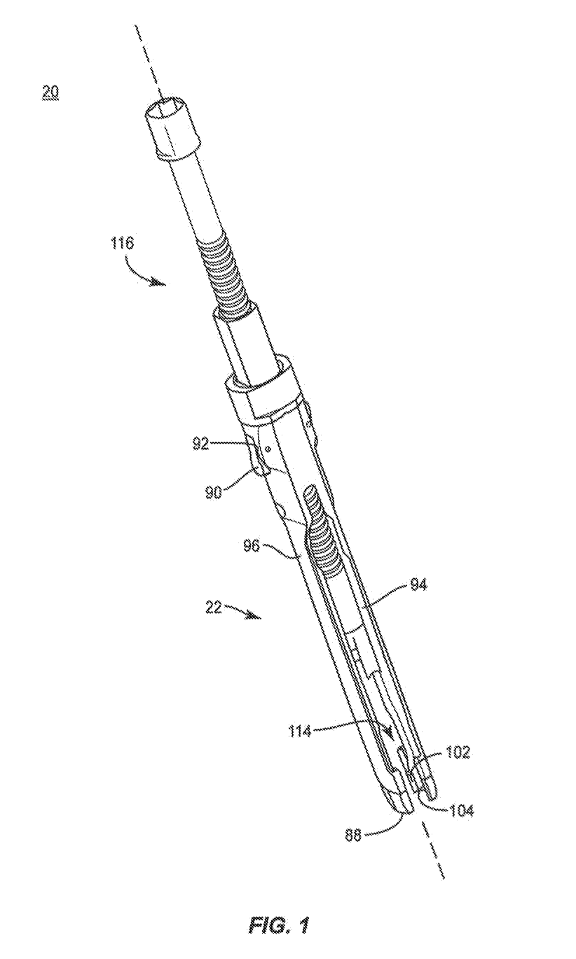

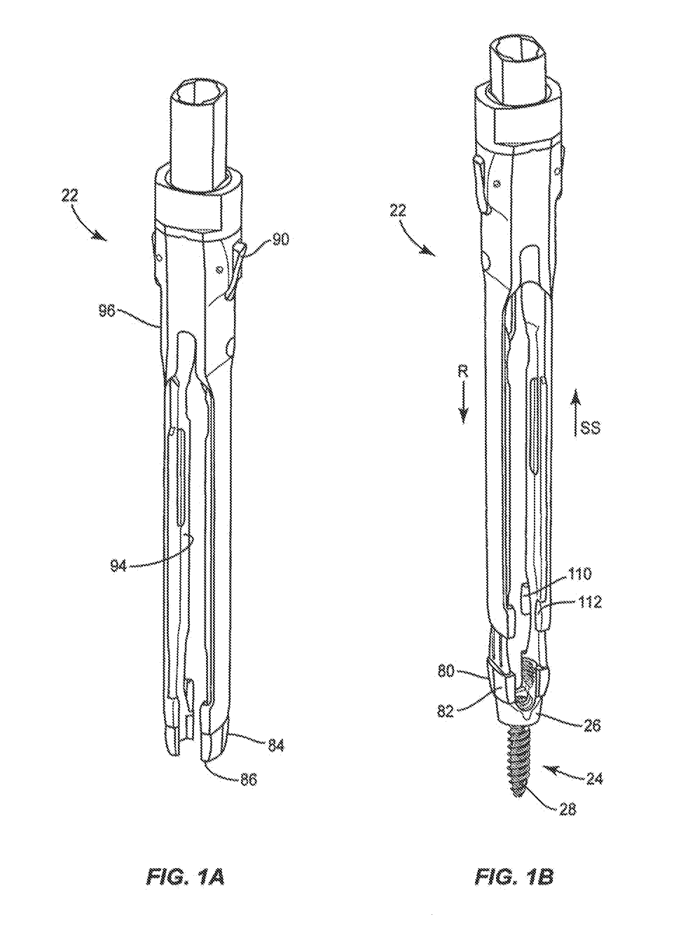

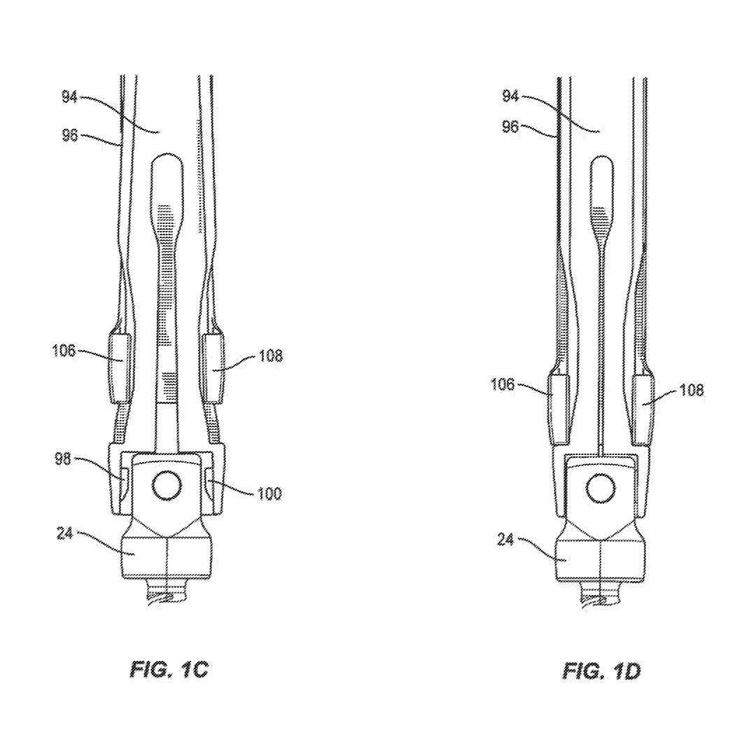

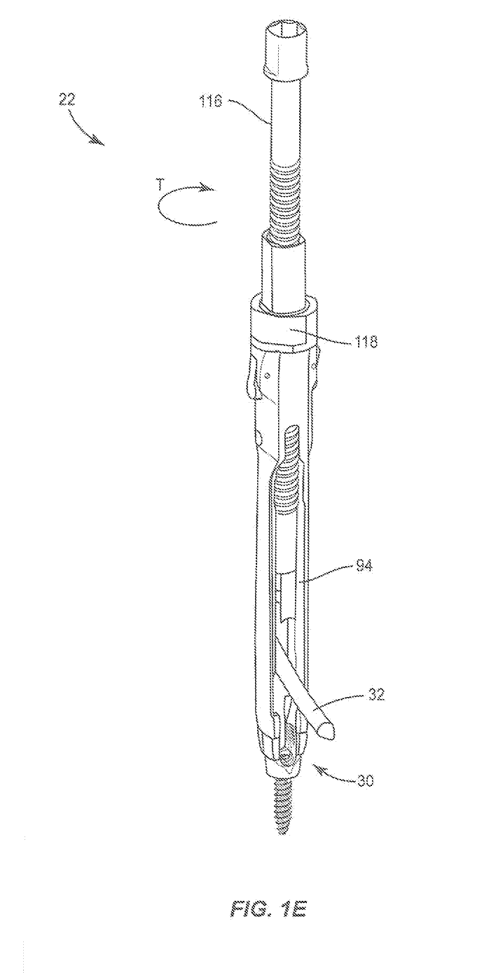

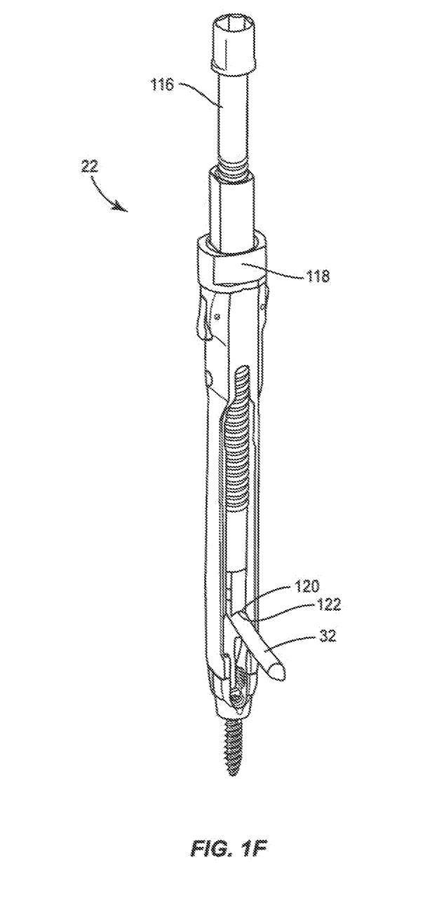

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning to FIGS. 1-15, there are illustrated components of a surgical system, such as, for example, a spinal implant system 20.

The components of spinal implant system 20 can be fabricated from biologically acceptable materials suitable for medical applications, including metals, synthetic polymers, ceramics and bone material and/or their composites. For example, the components of spinal implant system 20, individually or collectively, can be fabricated from materials such as stainless steel alloys, commercially pure titanium, titanium alloys, Grade 5 titanium, superelastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal implant system 20 may have material composites, including the above materials, to achieve various desired characteristics such as strength, rigidity, elasticity, compliance, biomechanical performance, durability and radiolucency or imaging preference. The components of spinal implant system 20, individually or collectively, may also be fabricated from a heterogeneous material such as a combination of two or more of the above-described materials. The components of spinal implant system 20 may be monolithically formed, integrally connected or include fastening elements and/or instruments, as described herein.

Spinal implant system 20 is employed, for example, with a minimally invasive procedure, including percutaneous techniques, and mini-open surgical techniques to deliver and introduce instrumentation and/or an implant, such as, for example, a bone fastener, at a surgical site within a body of a patient, which includes, for example, a spine having vertebrae.

Spinal implant system 20 includes a surgical instrument, such as, for example, an extender 22, as shown in FIGS. 1-1F. Extender 22 is a single, multi-functional surgical instrument and is configured for treating multiple pathologies, as described herein. Extender 22 is adaptable and includes a mating element, as described herein, for connection to a mating element of one or a plurality of alternate surgical instruments in a configuration for treating one or multiple pathologies, as described herein. In some embodiments, the alternate surgical instruments may comprise various surgical instruments, such as, for example, drivers, extenders, reducers, spreaders, distractors, blades, clamps, forceps, elevators and drills, which may be alternately sized and dimensioned, and arranged as a kit. For example, extender 22 can be employed to treat degenerative disorders; degenerative and deformity disorders; trauma, tumor and infection disorders; and advanced deformity disorders, and any combination thereof. This configuration avoids the need for multiple and different extenders systems to address different pathologies.

In some embodiments, extender 22 can be employed to treat degenerative disorders such that the components of spinal implant system 20 engage spinal tissue along one or two vertebral levels; degenerative and deformity disorders such that the components of spinal implant system 20 engage spinal tissue along three or four vertebral levels and the spine may be coronally unbalanced and/or sagittaly unbalanced; trauma, tumor and infection disorders with and without anterior column support; and advanced deformity disorders such that the components of spinal implant system 20 engage spinal tissue along four or more vertebral levels and the spine is sagittaly unbalanced. In some embodiments, extender 22 can be employed to treat degenerative disorders such that the components of spinal implant system 20 engage spinal tissue along one or a plurality of vertebral levels.

Extender 22 is configured for connecting a bone fastener 24 with vertebrae V, as described herein. In some embodiments, one or more of bone fasteners 24 may be engaged with tissue in various orientations, such as, for example, series, parallel, offset, staggered and/or alternate vertebral levels. In some embodiments, one or more of bone fasteners 24 may comprise multi-axial screws, sagittal angulation screws, pedide screws, monoaxial screws, uni-planar screws, facet screws, fixed screws, tissue penetrating screws, conventional screws, expanding screws, wedges, anchors, buttons, dips, snaps, friction fittings, compressive fittings, expanding rivets, staples, nails, adhesives, posts, fixation plates and/or posts.

Each of bone fasteners 24 comprise a first portion, such as, for example, a receiver 26 and a second portion, such as, for example, an elongated shaft 28 configured for penetrating tissue. Receiver 26 includes a pair of spaced apart arms having an inner surface that defines a U-shaped passageway 30. Passageway 30 is configured for disposal of a longitudinal element, such as, for example, a spinal rod 32. In some embodiments, the arms of receiver 26 may be disposed at alternate orientations, relative to shaft 28, such as, for example, those alternatives described herein.

In one embodiment, one or more of bone fasteners 24 may have a multi axial configuration such that the receiver is rotatable to a selected angle through and within an angular range to capture a spinal rod for fixation therein. The inner surface of receiver 26 includes a thread form configured for engagement with a coupling member, such as, for example, a set screw. The set screw is threaded with receiver 26 to attach, provisionally fix and/or lock spinal rod 32 with at least one of bone fasteners 24.

Shaft 28 has a cylindrical cross section configuration and includes an outer surface having an external thread form. In some embodiments, the external thread form may include a single thread turn or a plurality of discrete threads. In some embodiments, other engaging structures may be located on shaft 28, such as, for example, a nail configuration, barbs, expanding elements, raised elements and/or spikes to facilitate engagement of shaft 28 with tissue.

In some embodiments, all or only a portion of shaft 28 may have alternate cross section configurations, such as, for example, oval, oblong, triangular, square, polygonal, irregular, uniform, non-uniform, offset, staggered, undulating, arcuate, variable and/or tapered. In some embodiments, the outer surface of shaft 28 may include one or a plurality of openings. In some embodiments, all or only a portion of the outer surface of shaft 28 may have alternate surface configurations, such as, for example, smooth and/or surface configurations to enhance fixation with tissue, such as, for example, rough, arcuate, undulating, porous, semi-porous, dimpled, polished and/or textured. In some embodiments, all or only a portion of shaft 28 may be cannulated.

Spinal rod 32 has a cylindrical cross section configuration. In some embodiments, spinal implant system 20 may include one or a plurality of spinal rods 32, which may be relatively disposed in a side by side, irregular, uniform, non-uniform, offset and/or staggered orientation or arrangement. In some embodiments, spinal rod 32 can have a uniform thickness/diameter. In some embodiments, spinal rod 32 may have various surface configurations, such as, for example, rough, threaded for connection with surgical instruments, arcuate, undulating, dimpled, polished and/or textured. In some embodiments, the thickness defined by spinal rod 32 may be uniformly increasing or decreasing, or have alternate diameter dimensions along its length. In some embodiments, spinal rod 32 may have various cross section configurations, such as, for example, oval, oblong, triangular, rectangular, square, polygonal, irregular, uniform, non-uniform, variable and/or tapered. In some embodiments, spinal rod 32 may have various lengths. In some embodiments, the longitudinal element may include one or a plurality of tethers.

In some embodiments, the longitudinal element may have a flexible configuration and fabricated from materials, such as, for example, polyester, polyethylene, fabric, silicone, polyurethane, silicone-polyurethane copolymers, polymeric rubbers, polyolefin rubbers, elastomers, rubbers, thermoplastic elastomers, thermoset elastomers and elastomeric composites. In one embodiment, the flexibility of the longitudinal element includes movement in a lateral or side to side direction and prevents expanding and/or extension in an axial direction. In some embodiments, all or only a portion of the longitudinal element may have a semi-rigid, rigid or elastic configuration, and/or have elastic properties, such as the elastic properties corresponding to the material examples described above. In some embodiments, the longitudinal element may be compressible in an axial direction. In some embodiments, the implant can include spinal constructs, such as, for example, bone fasteners, spinal rods, connectors and/or plates. In other embodiments, various components of the spinal implant system 20 may also be utilized in open or traditional spinal surgical techniques.

In some embodiments, spinal implant system 20 may comprise the use of microsurgical and image guided technologies, such as, for example, surgical navigation components employing emitters and sensors, which may be employed to track introduction and/or delivery of the components of spinal implant system 20 including the surgical instruments to a surgical site. See, for example, the surgical navigation components and their use as described in U.S. Pat. Nos. 6,021,343, 6,725,080, 6,796,988, the entire contents of each of these references being incorporated by reference herein.

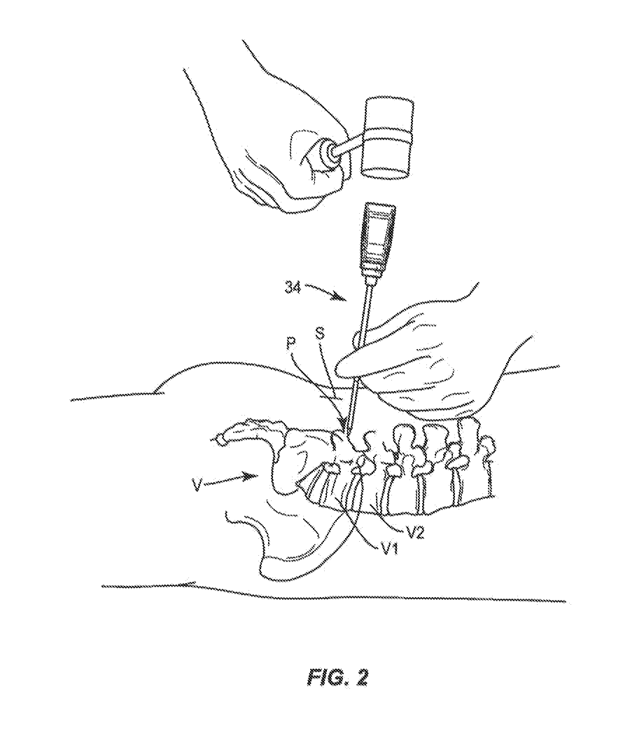

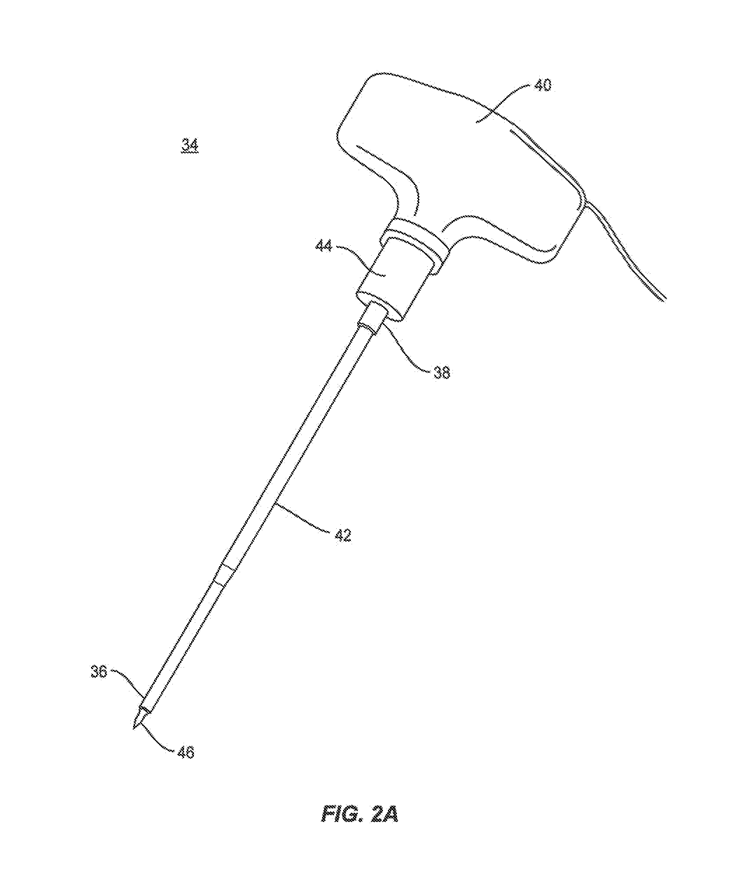

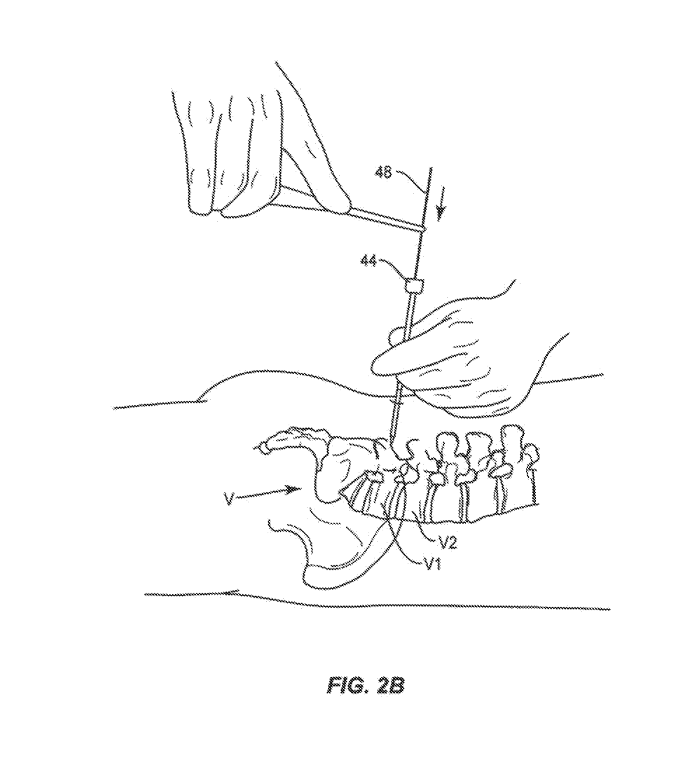

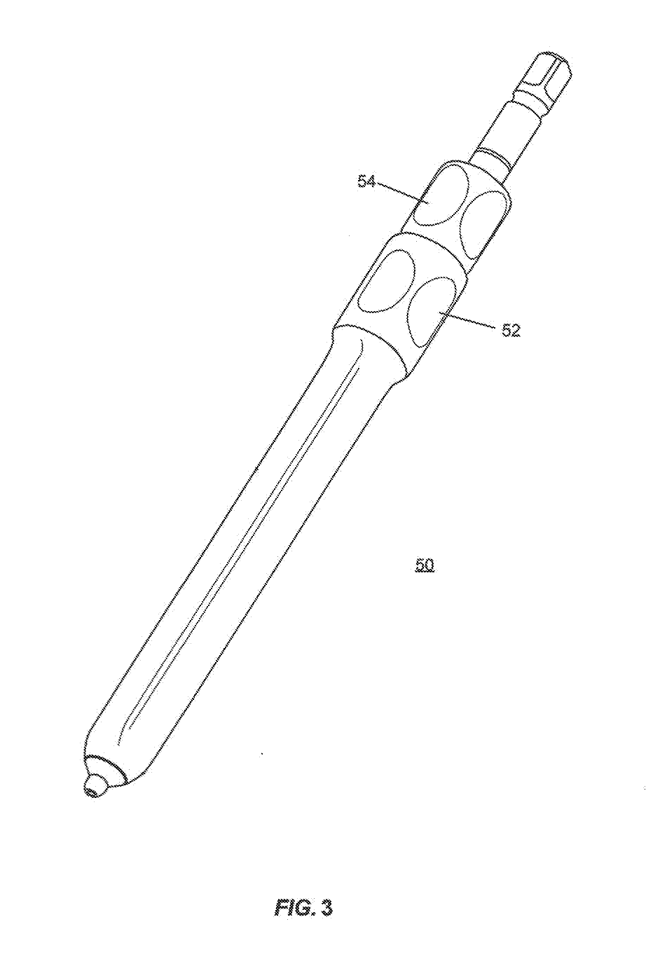

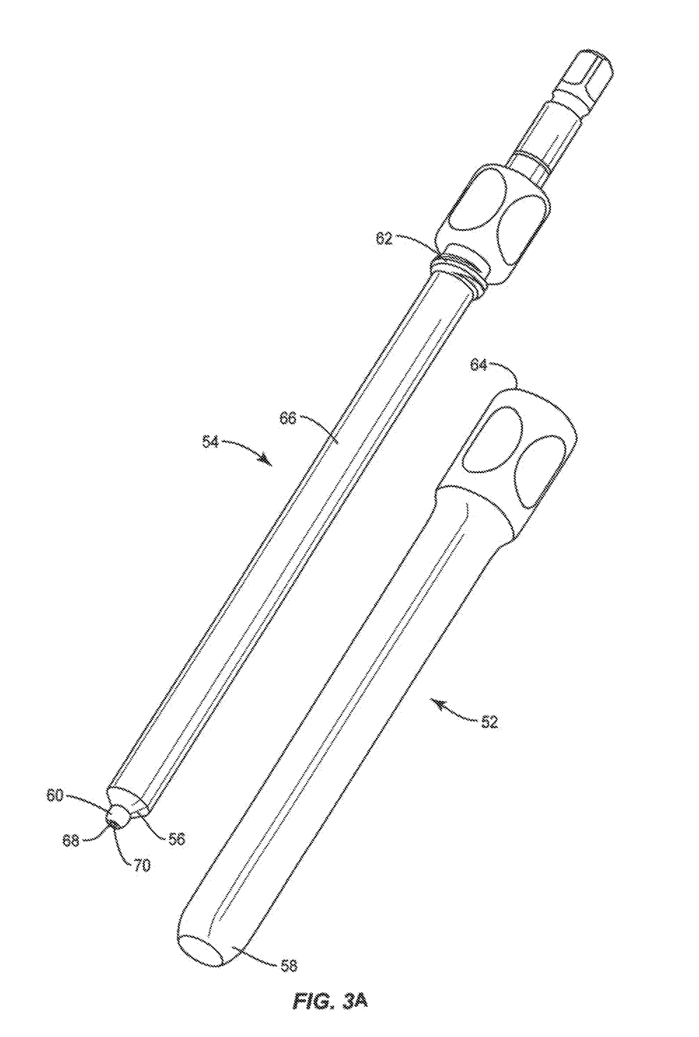

In assembly, operation and use, spinal implant system 20 is employed with a surgical procedure for treatment of a spinal disorder affecting a section of a spine of a patient, as discussed herein. Spinal implant system 20 may also be employed with other surgical procedures. For example, spinal implant system 20 can be used with a surgical procedure for treatment of a condition or injury of an affected section of the spine including vertebrae V, as shown in FIGS. 2 and 2B.

In use, to treat the affected section of vertebrae V, a medical practitioner obtains access to a surgical site including vertebral levels V1, V2 in any appropriate manner, such as through incision and retraction of tissues. In some embodiments, spinal implant system 20 may be used in a mini-open surgery, minimally invasive surgery, and percutaneous surgical implantation, whereby vertebrae is accessed through a micro-incision, or sleeve that provides a protected passageway to the area. Once access to the surgical site is obtained, the particular surgical procedure is performed for treating the spinal disorder. Spinal implant system 20 is then employed to augment the surgical treatment. Spinal implant system 20 can be delivered or implanted as a pre-assembled device or can be assembled in situ. Spinal implant system 20 may be completely or partially revised, removed or replaced during or after the surgical procedure.

In some embodiments, spinal implant system 20 is employed for treating degenerative disorders such that the components of spinal implant system 20 engage spinal tissue along one or two vertebral levels. In some embodiments, spinal implant system 20 is employed for treating degenerative disorders such that the components of spinal implant system 20 engage spinal tissue along a plurality of vertebral levels. In some embodiments, the degenerative disorders include degenerative disc disease, such as, for example, back pain of discogenic origin with degeneration of the disc, spondylolisthesis and spinal stenosis.

In one example, spinal implant system 20 can be employed for a one vertebral level spondylolisthesis reduction application. In some embodiments, a spinal needle may be used to verify the appropriate location of the skin incisions. The needle is positioned on the skin directly over a pedide of vertebral level V1 and medical imaging, such as AP and lateral images, confirms skin incision location. Microsurgical and image guided technologies, as described herein, can provide assistance with surgical instrument and implant navigation in a minimally invasive spinal fusion procedure.

In some embodiments, a complementary surgical instrument, such as, for example, a pedide access kit (PAK) needle 34 is used to gain access to vertebral tissue, as shown in FIGS. 2 and 2A, and create a surgical pathway P to the surgical site. Needle 34 is configured to gain access to vertebrae V. Needle 34 extends between a proximal end 36 and a distal end 38. End 38 includes a handle 40. A body 42 of needle 34 defines a cannula housing and an inner stylet 44 connected to handle 40. Needle 34 includes a tip 46 configured to puncture tissue. End 36 is placed at the intersection of a facet and a transverse process of vertebral level V1, and is advanced. In some embodiments, needle 34 may be advanced across the junction of a pedide of vertebral level V1 to allow easier placement of a guidewire 48, as shown in FIG. 2B.

Stylet 44 is removed to allow guidewire 48 to be inserted into the pedide. Guidewire 48 is connected and/or fastened with tissue of vertebral level V1. Guidewire 48 is disposed to extend along surgical pathway P from the surgical site adjacent vertebrae V to the incision for a percutaneous surgical procedure.

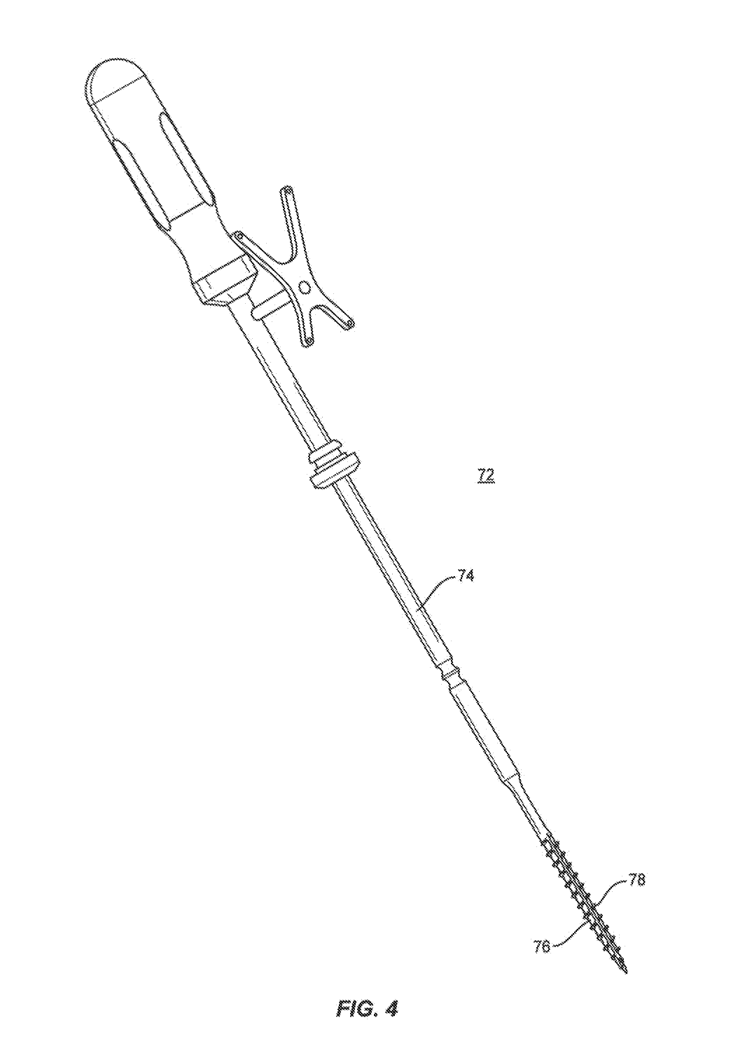

In some embodiments, as shown in FIGS. 3 and 3A, spinal implant system 20 includes a complementary surgical instrument, such as, for example, a surgical instrument 50 having a dilator 52 disposed with a dilator 54 for creating surgical pathway P and/or preparing a surgical site. An end 56 is disposed in a nested and mating configuration with an end 58. A probe tip 60 extends beyond end 58. A mating surface 62 engages a mating surface 64 and an outer surface 66 to releasably engage dilators 52, 54 for assembly thereof. A practitioner grips surface 66 and disposes guidewire 48 through an opening 68 and a passageway 70.

The practitioner manipulates instrument 50 to percutaneously navigate and deliver dilators 52, 54 along surgical pathway P from the incision to the surgical site adjacent vertebral level V1. Dilators 52, 54 are translated along guidewire 48 via passageway 70 such that probe tip 60 is introduced through the tissue and muscle leading to vertebral level V1. Initially probe tip 60 spaces apart and separates the tissue and muscle, and then surface 66 spaces apart and separates the tissue and muscle leading to the vertebra.

Upon disposal of probe tip 60 adjacent vertebral level V1, dilator 54 is removed from dilator 52. Dilator 54 is translated along guidewire 48 and delivered along surgical pathway P from the surgical site adjacent vertebral level V1 to the incision. Dilator 54 is removed from guidewire 48. In some embodiments, the pedide of vertebral level V1 is prepared by placing a surgical instrument over guidewire 48 and through dilator 52. Guidewire 48 is disposed with a passageway of a complementary surgical instrument, such as, for example, a tap 72, as shown in FIG. 4.

Tap 72 is configured for movable disposal within passageway 70 such that dilator 54 and tap 72 are coaxial and tap 72 can simultaneously rotate and translate axially within passageway 70. Tap 72 includes a cylindrical shaft 74 having a proximal end 76 including a screw tap 78 including an external or male thread configured to form an internal or female thread in the tissue such that an implant, such as, for example, bone fastener 24, can be threaded into the internal thread formed by tap 72. In one embodiment, screw tap 78 includes a tapered thread to facilitate insertion of screw tap 78 into tissue. In one embodiment, screw tap 78 includes a self-starting thread.

Tap 72 is translated along guidewire 48 and delivered along surgical pathway P and disposed within passageway 70 at the surgical site adjacent vertebral level V1. Tap 72 is employed to create a cavity in vertebral level V1 for disposal of a bone fastener or screw. In some embodiments, instrument 50 and/or the non-implanted components of spinal implant system 20 are removed from the surgical site. In some embodiments, a bone fastener and/or screw is translated along guidewire 48 and delivered along surgical pathway P and disposed within passageway 70 at the surgical site adjacent vertebral level V1 for fastening with vertebral level V1. In some embodiments, spinal implant system 20 includes a series of sequential dilators and/or a retractor arrangement to space tissue or creating a surgical pathway and/or preparing a surgical site.

In some embodiments, tap 72 may be configured to engage an actuator, such as, for example, a surgical instrument, powered drill, hand drill, driver or other tool to rotate tap 72. Fluoroscopy can be used to verify the position of guidewire 48 and tap 72.

Extender 22 is delivered to the surgical site along surgical pathway P and adjacent the vertebrae. Extender 22 includes capturing elements 80, 82 and 84, 86. Surfaces of elements 80, 82 and 84, 86 are disposed in a flush contacting engagement in a non-expanded configuration, as shown in FIG. 1. Bone fastener 24 is disposed adjacent a distal end 88. A button 90 is depressed to overcome the resilient bias of a spring and to release a projection 92 from a corresponding depression on an inner sleeve 94. Sleeve 94 is manually translated, in a direction shown by arrow R in FIG. 1B, relative to an outer sleeve 96, to dispose sleeve 94 in an expandable configuration, as described herein. In some embodiments, an audible dick is heard to confirm that sleeves 94, 96 are in the expandable configuration.

Receiver 26 of bone fastener 24 is aligned with sleeve 94 such that elements 80, 82 and 84, 86 separate and expand for engagement and capture of bone fastener 24. Projections 98, 100 and 102, 104 are aligned and engaged with lock slots of bone fastener 24 to capture bone fastener 24.

Button 90 is depressed and sleeve 94 is manually translated, in the direction shown by arrow SS in FIG. 1B. As sleeve 94 translates, sleeve 94 contracts between an expanded configuration to a non-expandable configuration, as shown and described with regard to FIGS. 1C and 1D. In the non-expandable configuration, flanges 106, 108 and 110, 112 engage sleeve 94 to resist and/or prevent expansion of sleeve 94 and disengagement of bone fastener 24 from sleeve 94. In some embodiments, an audible dick is heard to confirm that sleeves 94, 96 are in the non-expandable configuration.

Extender 22 is fixed with bone fastener 24 and delivered adjacent vertebrae at a surgical site. In some embodiments, a surgical instrument, such as, for example, a driver is disposed with extender 22 and a cavity 114 to engage bone fastener 24. Shaft 28 is inserted, drilled or otherwise fixed to vertebrae to attach bone fastener 24 with vertebrae.

A reducer 116 includes a mating element, such as, for example, a threaded outer surface that engages a mating element, such as, for example, a projection of an inner surface of a knob 118 of extender 22. Reducer 116 comprises an interchangeable surgical instrument that connects with extender 22. Spinal rod 32 is disposed with cavity 114 for alignment and disposal with receiver 26, as shown in FIG. 1E. Reducer 116 is inserted into cavity 114. Knob 118 is movable to selectively reduce spinal rod 32 with bone fastener 24 between a quick reduction, such that reducer 116 is freely translatable relative to sleeve 96 and the projection does not engage the threaded outer surface of reducer 116, and a controlled reduction, such that the projection engages the threaded outer surface of reducer 116 and reducer 116 is incrementally translatable relative to sleeve 96. Spinal rod 32 is disposed with receiver 26 and a coupling member, such as, for example, a set screw, such that spinal rod 32 is fixed with bone fastener 24.

In some embodiments, knob 118 is rotatable in a clockwise direction, as shown by arrow T in FIG. 1E, to disengage a surface 120 and a section 122, and release reducer 116 from extender 22. Reducer 116 is translated, in the direction shown by arrow SS in FIG. 1B, relative to sleeve 96 to remove reducer 116 from extender 22. Projections 98, 100 and 102, 104 disengage the lock slots of receiver 26 to eject bone fastener 24 from sleeve 94.

Extender 22 is removed from bone fastener 24. The final construct can be verified with AP and lateral fluoroscopy. The entire process is repeated on the contralateral side of the vertebrae.

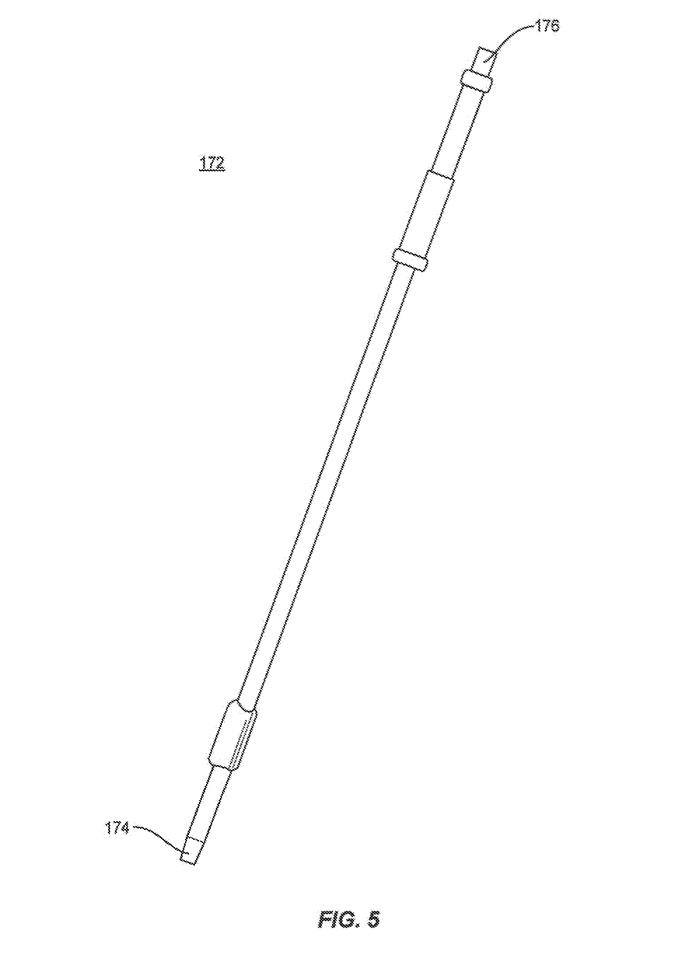

In some embodiments, a bonescrew driver is inserted with extender 22. Spinal implant system 20 includes a complementary surgical instrument, such as, for example, a driver 172, as shown in FIG. 5. Driver 172 is configured for engagement with a bone screw, such as, for example, bone fastener 24. Driver 172 extends between a proximal end 174 and a distal end 176. Driver 172 engages bone fastener 24, which includes surface structures configured to engage end 174 and is threaded into the cavity in vertebral level V1 created by screw tap 78.

A tip of driver 172 passes into receiver 26 of bone fastener 24 until driver 172 fully engages bone fastener 24. A sleeve of driver 172 is threaded into receiver 26 before inserting bone fastener 24 over guidewire 48. Extender 22 is inserted over guidewire 48 and into the pedide of vertebral level V1. After driving bone fastener 24 through the pedide, guidewire 48 is removed to prevent it from being advanced. The process is repeated for additional bone fasteners 24 on the same side.

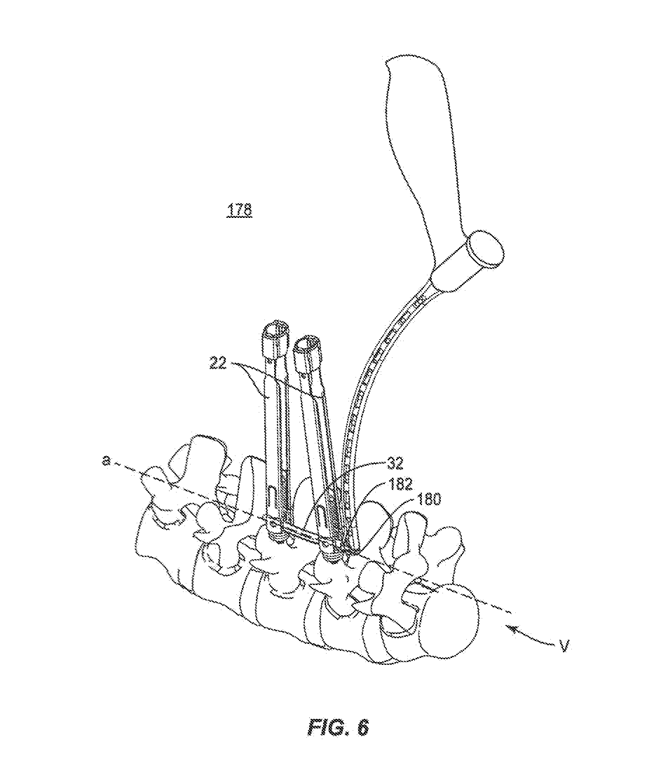

For example, in some embodiments, spinal implant system 20 includes a complementary surgical instrument, such as, for example, a rod inserter 178, as shown in FIG. 6, configured for engagement with a spinal construct, which includes, for example, spinal rod 32. Rod inserter 178 includes jaws 180, 182 disposed adjacent spinal rod 32 in a non-locking and open orientation. In some embodiments, a latch is rotated to a locking orientation to close jaws 180, 182 and capture spinal rod 32. In some embodiments, rod inserter 178 may comprise an alternate, interchangeable surgical instrument having a mating element, such as, for example, an outer surface of jaws 180, 182 that is engageable with a mating element, such as, for example, an outer surface of sleeve 94 of extender 22.

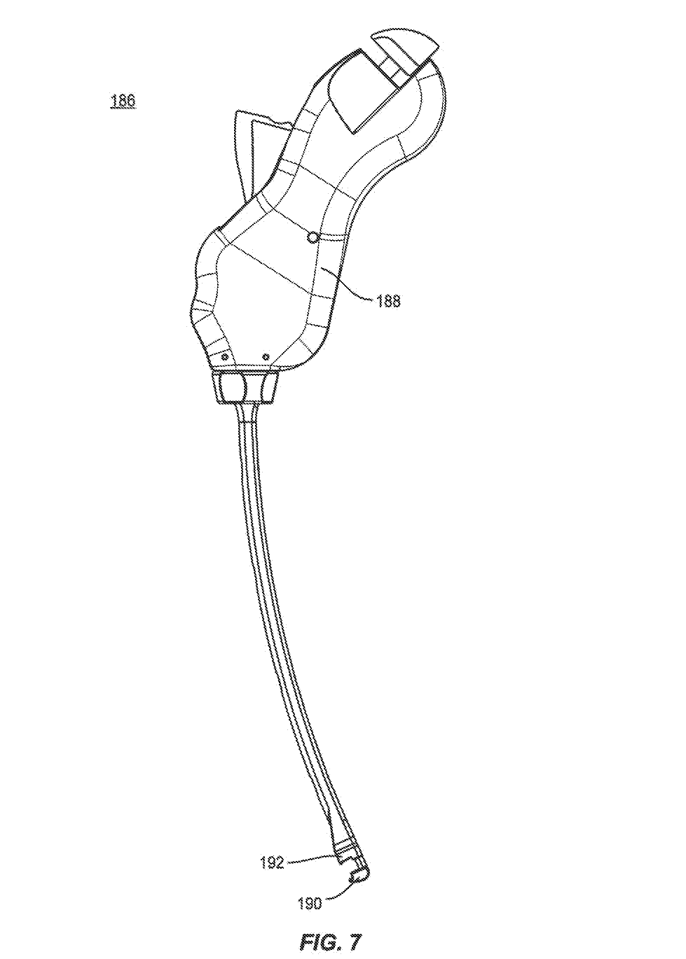

In one embodiment, as shown in FIG. 7, a practitioner employs a surgical instrument 186 for insertion of spinal rod 32 through extenders 22 along a path that includes a distal and/or far location from the practitioner of the path corresponding to an adjacent bone fastener, to a proximal and/or near location to the practitioner of the path corresponding to an adjacent bone fastener. Spinal rod 32 is disposed with instrument 186 in a locking orientation.

The practitioner grips a handle 188 of surgical instrument 186 to dispose a distal tip of spinal rod 32 inside a window of extender 22 and above the skin and fascia. Instrument 186 orients spinal rod 32 such that an axis a of spinal rod 32 is offset from an angle of extender 22. The angle is disposed in a first plane, such as, for example, a sagittal plane SP (not shown) of vertebrae V. In some embodiments, the angle is oriented in a cephalad-caudal orientation. In some embodiments, surgical instrument 186 may comprise an alternate, interchangeable surgical instrument having a mating element, such as, for example, an outer surface of jaws 190, 192 that is engageable with a mating element, such as, for example, an outer surface of sleeve 94 of extender 22. In some embodiments, instrument 186 orients spinal rod 32 such that axis a is offset an angle disposed in a second plane, such as, for example, a transverse plane TP (not shown) of vertebrae V.

With the distal tip of spinal rod 32 disposed beneath the skin and fascia and adjacent extender 22, instrument 186 orients spinal rod 32 to simultaneously lower spinal rod 32 to receiver 26 of bone fastener 24 and rotate spinal rod 32 through extenders 22. Instrument 186 orients spinal rod 32 to lower spinal rod 32 with the receivers 26 of bone fasteners 24. Instrument 186 includes jaws 190, 192 which have a greater width dimension than the window of extender 22 and are prevented from entering the window of extender 22 and/or receiver 26. In some embodiments, one or more of extenders 22 are configured for engagement with a reduction instrument to engage spinal rod 32 and reduce spinal rod 32 into receivers 26 of bone fasteners 24.

In another example, in one embodiment, similar to the method described above with regard to FIGS. 1-7, a practitioner employs instrument 186 for insertion of spinal rod 32 through extenders 22 along a path that includes a proximal and/or near location to the practitioner of the path corresponding to an adjacent bone fastener 24, to a distal and/or far location from the practitioner of the path corresponding to an adjacent bone fastener 24. Spinal rod 32 is disposed with instrument 186 in the locking orientation, as described.

The practitioner grips handle 188 to dispose the distal tip of spinal rod 32 inside the window of extender 22 and within receiver 26. Instrument 186 orients spinal rod 32. With the distal tip of spinal rod 32 disposed beneath the skin and fascia and adjacent extender 22, instrument 186 orients spinal rod 32 to simultaneously lower spinal rod 32 to receiver 26 of bone fastener 24 and rotate spinal rod 32 through extenders 22. Instrument 186 orients spinal rod 32 to lower spinal rod 32 with receiver 26 of bone fasteners 24. Spinal rod 32 is released from jaws 190, 192. Spinal rod 32 is locked with bone fasteners 24 by coupling members, such as, for example, set screws.

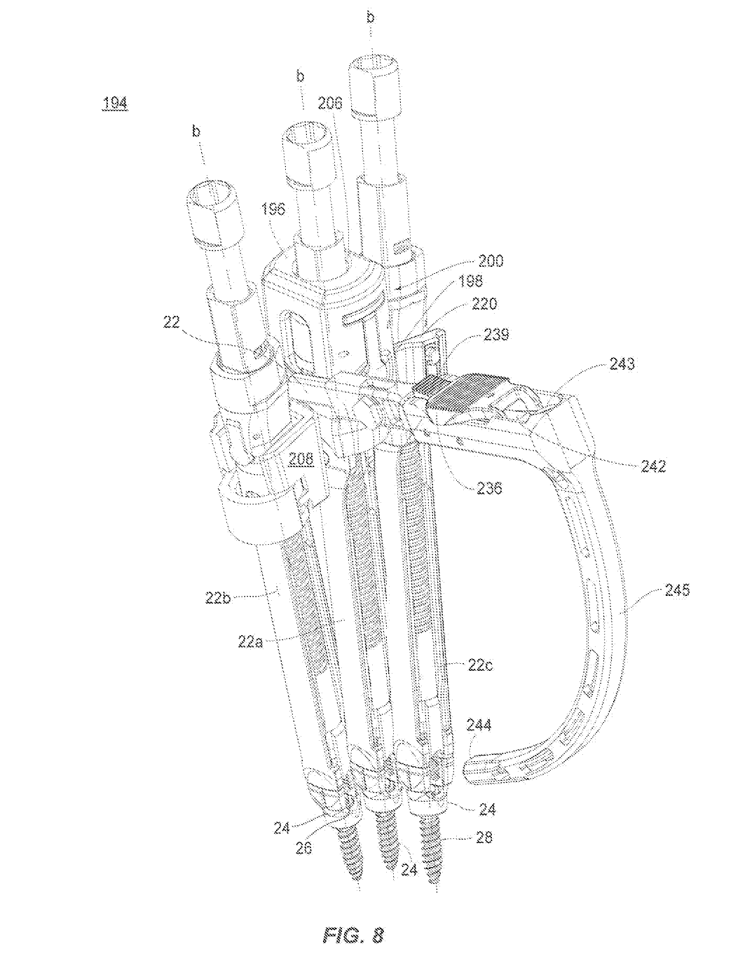

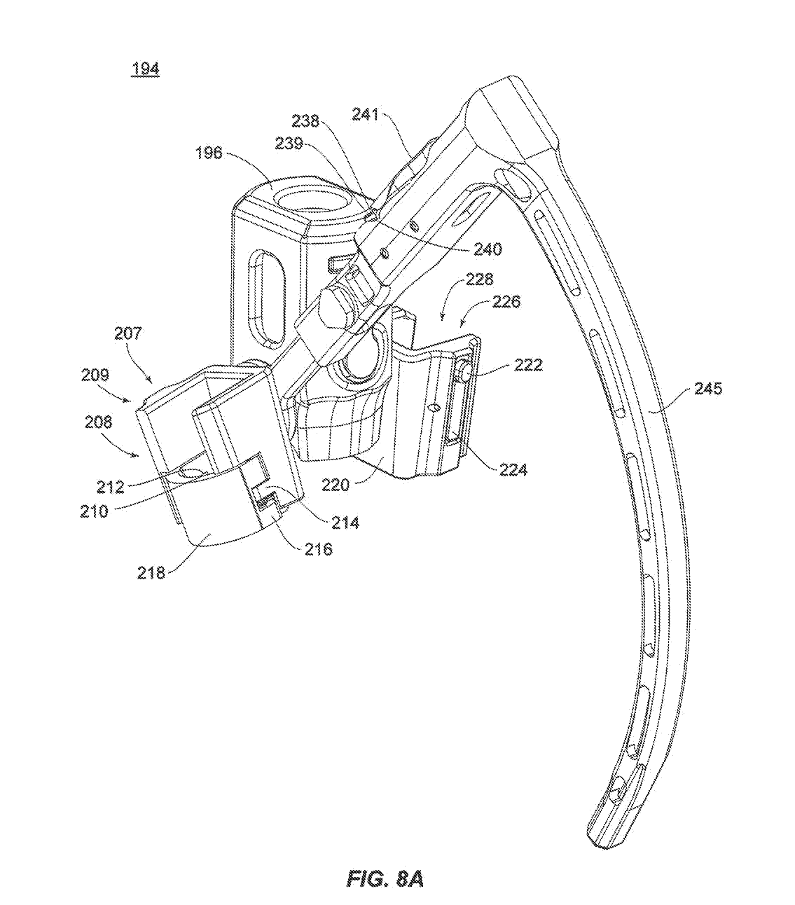

In one embodiment, as shown in FIGS. 8 and 8A, an alternate, interchangeable surgical instrument 194 with extender 22 includes a central housing 196 having a mating element, such as, for example, an inner surface thereof engageable with a mating element, such as, for example, an outer surface of a central extender 22a. A button 198 of a latch 200 is depressed and engages a spring (not shown), causing a prong (not shown) to engage latch 200, engaging and locking central extender 22a within a passage 206.

A housing 208, disposed laterally relative to central housing 196, is positioned adjacent extender 22b, which is disposed laterally relative to central extender 22a. Extender 22b is rotated in a counterclockwise direction. A button 207 of latch 209 is depressed and a tab 210 disengages a slot 212. A hinge 214 rotates an end 216 of a capture element 218 in a counterclockwise direction, which moves capture element 218 in an outward direction. Extender 22b is inserted into housing 208 and hinge 214 rotates end 216 of capture element 218 in a clockwise direction, which moves capture element 218 in an inward direction. Slot 212 engages tab 210 and an audible click is provided.

A housing 220, disposed laterally relative to central housing 196 is positioned adjacent extender 22c, which is disposed laterally relative to central extender 22a. Extender 22c is rotated in a clockwise direction. A button 222 of a latch 224 is depressed and a tab 226 disengages a slot 228. A hinge 230 (not shown) rotates an end 232 (not shown) of a capture element 234 (not shown) in a counterclockwise direction, which moves capture element 234 in an outward direction. Extender 22c is inserted into housing 220 and hinge 230 rotates end 232 of capture element 234 in a clockwise direction, which moves capture element 234 in an inward direction. Slot 228 engages tab 226 and an audible click is provided.

A lock 236 is rotated. An end 238 of a latch 239 disengages from a groove 240 of a lever 241. Lever 241 is rotated in a clockwise direction translating a biasing member 242 in a distal direction opening a collet 243. Spinal rod 32 is inserted into an end 244 and lever 241 is rotated in a counterclockwise direction translating biasing member 242 in a proximal direction, closing collet 243 such that rod 32 engages end 244. Lock 236 is rotated and engages groove 240. A member 245 is rotatable relative to one or more housings 196, 208, 220, to dispose rod 32 with extenders 22 and to place rod 32 in alignment with one or more bone fasteners 24. Instrument 194 aligns and disposes rod 32 with bone fasteners 24.

Spinal rod 32 is reduced with receivers 26 of bone fasteners 24. A reducer is translated such that an end surface of the reducer engages spinal rod 32 in a configuration to move spinal rod 32 distally to drive spinal rod 32 into receivers 26. Reducers are manipulated to draw receivers 26 up to receive spinal rod 32. In some embodiments, the reducers may reduce spinal rod 32 with various bone fasteners 24, as described herein, incrementally, continuously to engagement with spinal rod 32 and/or to disengagement from spinal rod 32.

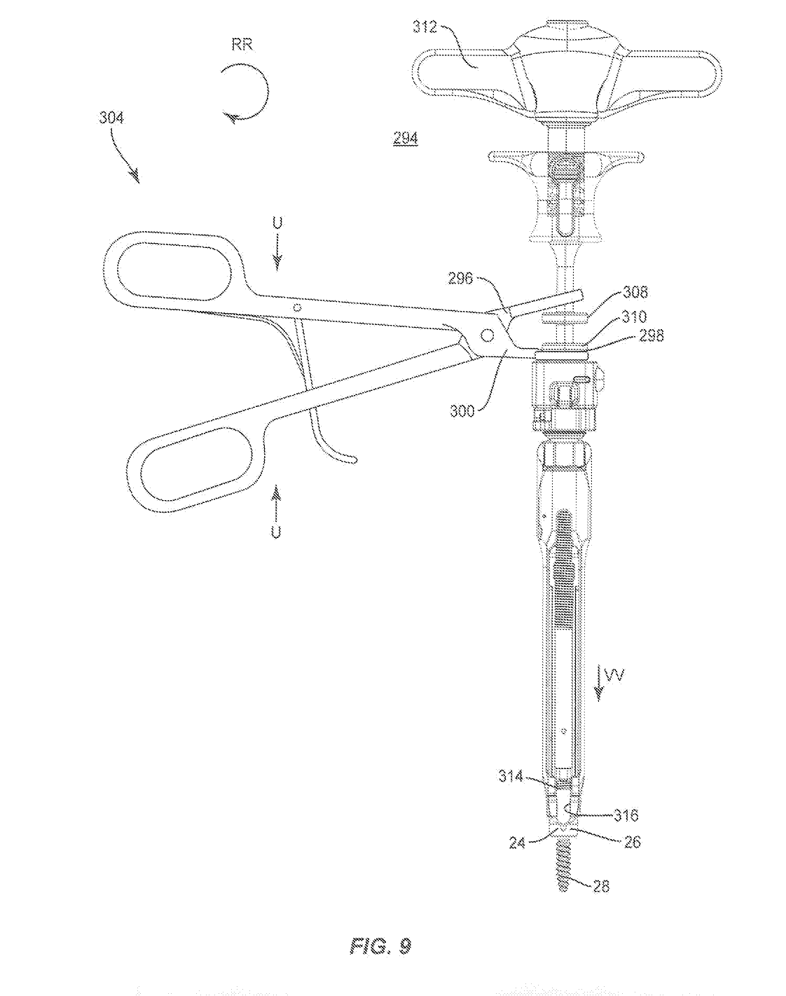

In some embodiments, as shown in FIG. 9, spinal implant system 20 includes an alternate, interchangeable surgical instrument, such as, for example, a rod reducer 294. Mating prongs 296 are engaged with a mating surface that defines a cavity 298 and prongs 300 are disposed in cavity 298. Pliers 304 are squeezed, as shown by arrows U, such that prongs 296 engage flanges 308, 310 to apply an axially directed force to a driver 312. Pliers 304 translate bone fastener 24, in the direction shown by arrow W, such that a thread form 314 engages a surface 316 in a second orientation. Driver 312 is rotated in the clockwise direction, as shown by arrow RR, to threadably engage thread form 314 with surface 316 to reduce spinal rod 32 into receivers 26 of bone fastener 24.

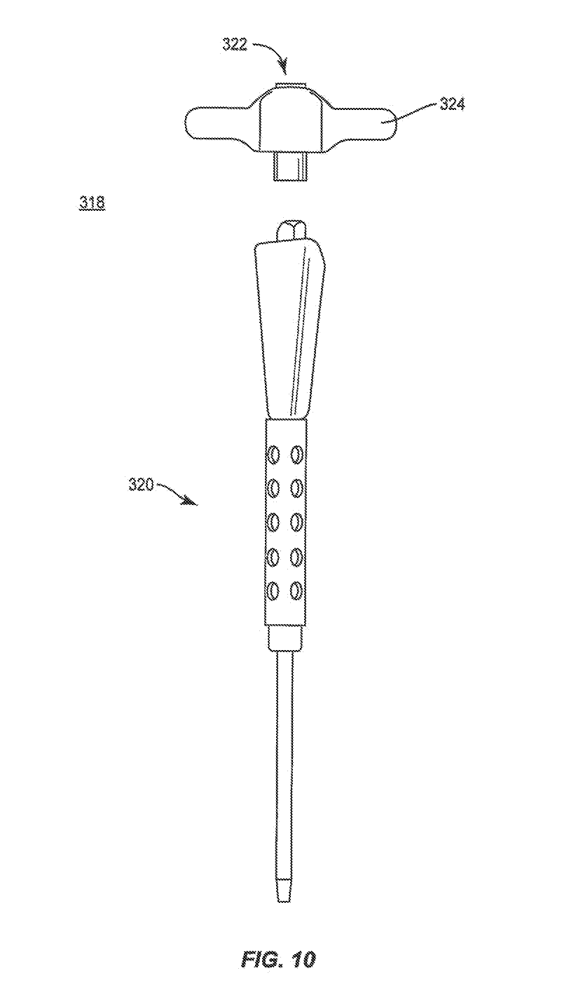

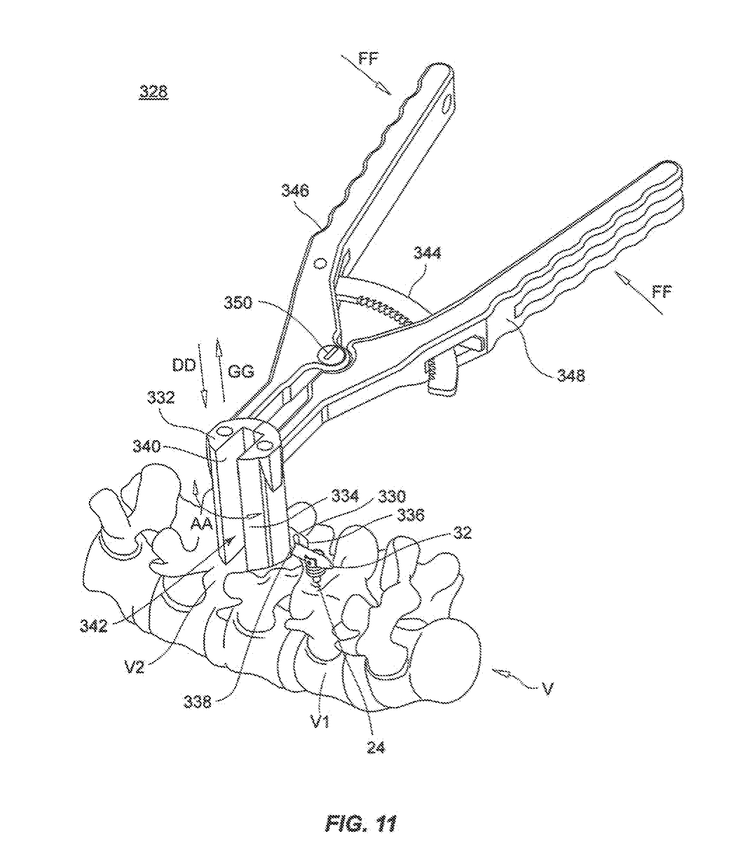

In some embodiments, as shown in FIG. 10, spinal implant system 20 includes compressor 318 that comprises an alternate, interchangeable surgical instrument with extender 22. To load a set screw 320, a button 322 is pressed on a compressor handle 324 and set screw 320 is attached to the working end of extender 22. Button 322 is released to secure the connection. In some embodiments, compressor 318 acts as a compressor or distractor, and/or a self-retaining break-off driver. Set screw 320 is loaded on a tip of compressor 318. Compressor 318 and set screw 320 are inserted down the extender shaft and set screw 320 is threaded into receiver 26 of bone fastener 24 to engage rod 32.

Set screw 320 is provisionally tightened. In one embodiment, two compressors 318 are squeezed together and lock down a final set screw 320 to compress. Two handles are pulled apart and lock down set screw 320 to distract. In some embodiments, the assembly serves as the counter-torque device during final break-off. In some embodiments, a break-off handle is used for the final break-off and provisionally tightens set screw 320. In some embodiments, compressor 318 retains a sheared-off portion of set screw 320.

In some embodiments, spinal implant system 20 is further employed to treat vertebrae V in connection with a surgical procedure, for example, those treatments described herein, and may include a surgical instrument used to facilitate spinal fusion, such as, for example, postero-lateral spinal fusion. In some embodiments, the components of spinal implant system 20 are configured to position an implant, such as, for example, bone graft with the transverse process(es) of the spine to achieve a postero-lateral fusion for treatment, as described herein.

For example, in some embodiments, as shown in FIG. 11, a complementary surgical instrument, such as, for example, a speculum 328 is oriented relative to vertebrae V such that an engagement part 330 is disposed adjacent spinal rod 32. A force is applied to speculum 328, in the direction shown by arrow DD, such that engagement part 330 and spinal rod 32 engage. Engagement part 330 engages and is disposed onto spinal rod 32 so that engagement part 330 is expandable from a non-expanded configuration to an expanded configuration, as described herein, for disposal of spinal rod 32 with blade members 332, 334. Engagement part 330 is disposed in a non-expanded configuration due to the spring bias of portions 336, 338 to a contracted orientation.

Spinal rod 32 engages an inner surface 340 adjacent a cavity 342 to overcome the bias of portions 336, 338. In the expanded configuration, portions 336, 338 expand such that spinal rod 32 is received within expanded cavity 342 and surface 340 engages an outer surface of spinal rod 32 in a snap-fit engagement such that engagement part 330 captures spinal rod 32 in a releasable engagement.

In some embodiments, a ratchet is disposed in the non-locking orientation, as described herein, and a force is applied to handle portions 346, 348, in a direction shown by arrows FF, to overcome the resilient bias of a spring 350 such that blade members 332, 334 move from the first orientation, to the second orientation, in the locking orientation. The expansion of blade members 332, 334, in the direction shown by arrows AA, applies a force on surrounding tissue to space the tissue and provide access and visualization of the surgical site to the practitioner. Speculum 328 spaces tissue and maintains a connection to spinal rod 32 via engagement part 330 for treating one or more vertebral levels, as described herein. In some embodiments, engagement part 330 may slidably engage the outer surface of spinal rod 32 to selectively position speculum 328.

In some embodiments, speculum 328 can be disconnected from spinal rod 32 and relocated to other working vertebral level(s) of vertebrae V for spacing tissue to provide access and visualization of the surgical site for treating the other working vertebral levels. To disengage spinal rod 32 from engagement part 330, a force is applied to speculum 328, in a direction shown by arrow GG. Surface 340 disengages the outer surface of spinal rod 32 and spinal rod 32 exits cavity 342. The spring bias of portions 336, 338 causes portions 336, 338 to contract such that portion 336 rotates toward portion 338 and engagement part 330 is disposed in the non-expanded configuration, as described herein. Upon completion of a procedure, the surgical instruments, assemblies and non-implanted components of spinal implant system 20 are removed and the incisions are dosed.

In some embodiments, a discectomy is performed adjacent an intervertebral space via surgical pathway P. In some embodiments, sequential trial implants are delivered along surgical pathway P and used to distract the intervertebral space and apply appropriate tension in a selected intervertebral space allowing for indirect decompression.

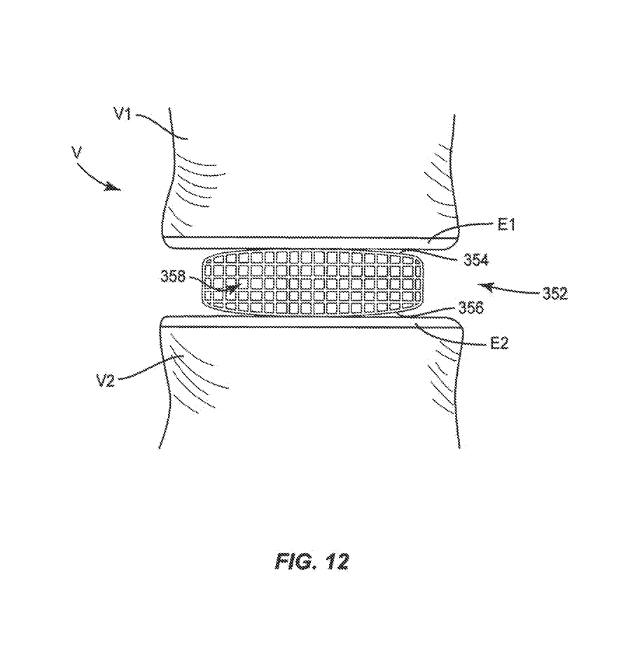

In some embodiments, trialing is utilized to establish a starting point for insertion of an interbody implant. In some embodiments, as shown in FIG. 12, spinal implant system 20 includes an implant, such as, for example, an interbody cage 352. Cage 352 includes a vertebral engaging surface 354 and a vertebral engaging surface 356. Surface 354 is configured to engage endplate tissue of a vertebral body, such as, for example, an endplate E1 of vertebral level V1. Surface 356 is configured to engage endplate tissue of a vertebral body, such as, for example, an endplate E2 of vertebral level V2. In some embodiments, surfaces 354, 356 may be rough, textured, porous, semi-porous, dimpled, knurled, toothed, grooved and/or polished such that it facilitates engagement with tissue. In some embodiments, the vertebral tissue may include intervertebral tissue, endplate surfaces and/or cortical bone. In one embodiment, an interbody cage 352, described herein, may be formed substantially of biocompatible polymer, such as PEEK, and selectively coated with a biocompatible metal, such as titanium, or a bone-growth promoting material, such as HA. In some such embodiments, titanium may be plasma sprayed onto surfaces of interbody cage 352 so as to modify the radiographic signature of cage 352 and/or improve the prospects of bony ongrowth to cage 352 by virtue of the application of a porous or semi-porous coating of titanium.

Cage 352 may be provided with a substantially cylindrical cross section configuration and includes an inner surface 358 that defines an opening configured to receive an agent, which may include bone graft and/or other materials, as described herein, for employment in a fixation or fusion treatment. In some embodiments, the cross-sectional geometry of cage 352 may have various configurations, such as, for example, round, oval, oblong, triangular, polygonal having planar or arcuate side portions, irregular, uniform, non-uniform, consistent, variable, horseshoe shape, U-shape or kidney bean shape.

Upon completion of a procedure, described herein, the surgical instruments, assemblies and non-implanted components of spinal implant system 20 are removed and the incisions are dosed. One or more of the components of spinal implant system 20 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 20. In some embodiments, spinal implant system 20 may include one or a plurality of plates, connectors and/or bone fasteners for use with a single vertebral level or a plurality of vertebral levels.

In one embodiment, spinal implant system 20 includes an agent, which may be disposed, packed, coated or layered within, on or about the components and/or surfaces of spinal implant system 20. In some embodiments, the agent may include bone growth promoting material, such as, for example, bone graft to enhance fixation of the components and/or surfaces of spinal implant system 20 with vertebrae. In some embodiments, the agent may include one or a plurality of therapeutic agents and/or pharmacological agents for release, including sustained release, to treat, for example, pain, inflammation and degeneration.

In one embodiment, spinal implant system 20, similar to the systems described above, includes unilateral bone fasteners, such as, for example, facet screws (not shown) similar to the bone fasteners described above, and a bone plate (not shown), and is employed with a surgical procedure for treatment of a spinal disorder, similar to the methods described above.

In one embodiment, spinal implant system 20, similar to the systems and methods described herein with regard to FIGS. 1-12, is employed for treating a spine having degenerative and deformity disorders such that the spine is coronally unbalanced and sagittaly balanced and the components of spinal implant system 20 engage spinal tissue along three or four vertebral levels.

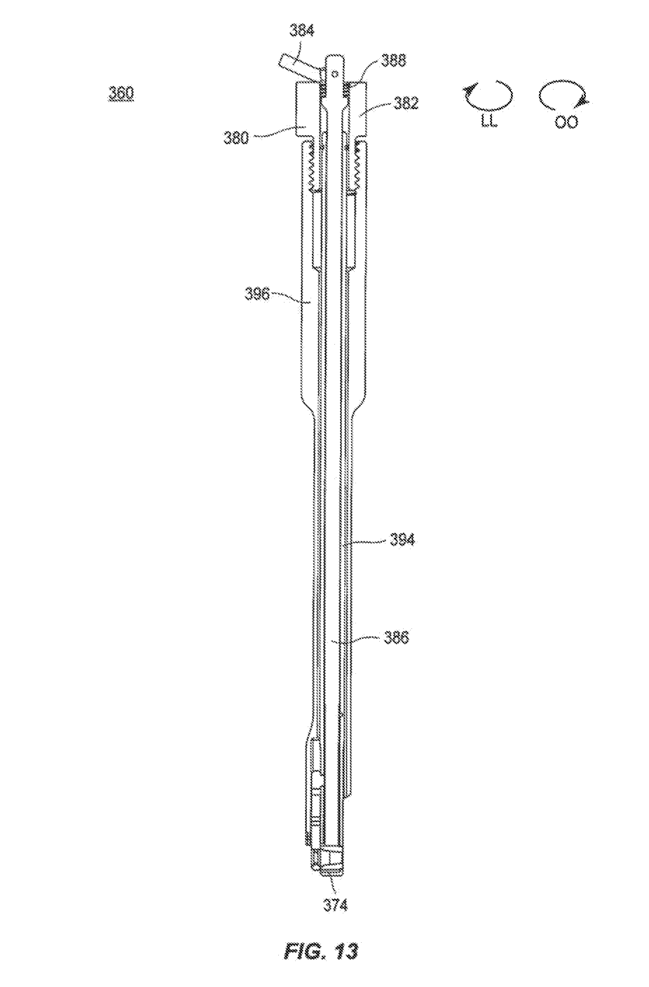

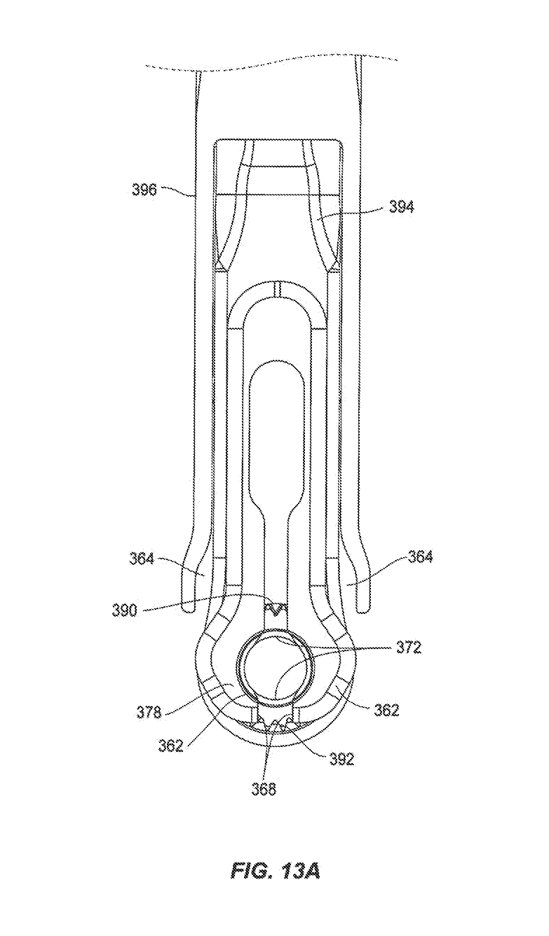

Upon fixation of bone fasteners 24 with four vertebral levels, similar to that described above, spinal rod 32 is positioned substantially orthogonal to a complementary surgical instrument, such as, for example, a rod rotator 360, as shown in FIGS. 13 and 13A. Jaws 362 are disposed in an open orientation, as described herein, such that jaws 362 are disengaged from concave end surfaces 364. In some embodiments, an end of spinal rod 32 is positioned with rod rotator 360 such that convex side portions of rod 32 engage concave sidewalls 368 and a planar top and bottom portions of rod 32 engage planar top and bottom walls 372 of a rotator 374. A recess disposed on spinal rod 32 mates with a flange 378 such that spinal rod 32 mates in a fixed configuration with jaws 362.

A portion 380 of an actuator 382 is rotated in a clockwise direction, as shown by arrow LL, to lock jaws 362 with spinal rod 32. Rotation of actuator 382 causes jaws 362 to move inwardly to tighten and lock spinal rod 32 with rod rotator 360, as described herein.

A lever 384 is disposed in a first position and rotator 374 is locked with a shaft 386, for delivering and insertion of rod rotator 360 adjacent to the surgical site. As such, the orientation of spinal rod 32 can be adjusted and manipulated with rod rotator 360, as described herein. To facilitate rotation of shaft 386 and rod rotator 360 relative to rotator 374 and spinal rod 32, lever 384 is depressed to actuate a spring 388 and disengage teeth 390 from teeth 392. In this configuration, rotator 374 and spinal rod 32 attached therewith can rotate relative to sleeves 394, 396 and rod rotator 360 to facilitate movement of rod rotator 360 and spinal rod 32 at the surgical site. This configuration facilitates manipulation and adjustment of the orientation of sleeves 394, 396 and/or rod rotator 360 without altering the position and orientation of spinal rod 32.

Actuator 382 is rotated in a counter-lockwise direction, as shown by arrow OO, to dispose jaws 362 in a non-locking orientation and release spinal rod 32 from rod rotator 360, as described herein. Upon completion of a procedure, the surgical instruments, assemblies and non-implanted components of spinal implant system 20 are removed and the incisions are closed.

In one embodiment, spinal implant system 20, similar to the systems and methods described herein with regard to FIGS. 1-13A, is employed for treating a spine having trauma, tumor and/or infection disorders with and without anterior column support. For example, spinal implant system 20 can be employed with a surgical correction treatment of an applicable condition or injury, such as, for example, a trauma of an affected section of a spinal column and adjacent areas within a body, such as, for example, a fractured vertebra of vertebrae. In some embodiments, spinal implant system 20 may be employed with one or a plurality of vertebra.

Pilot holes or the like are made in selected vertebra of the vertebrae adjacent the fractured vertebra for receiving bone fasteners 24a, b, with the fractured vertebra being disposed between adjacent vertebra. A driver is disposed adjacent the vertebrae at a surgical site and is manipulated to drive, torque, insert or otherwise connect bone fasteners 24a, b with the adjacent vertebra. Extenders 22a, 22b are delivered to the surgical site adjacent the vertebrae and oriented for manipulation, alignment and capture of bone fasteners 24a, 24b. Extender 22a is connected to bone fastener 24a and extender 22a is connected to bone fastener 24b.

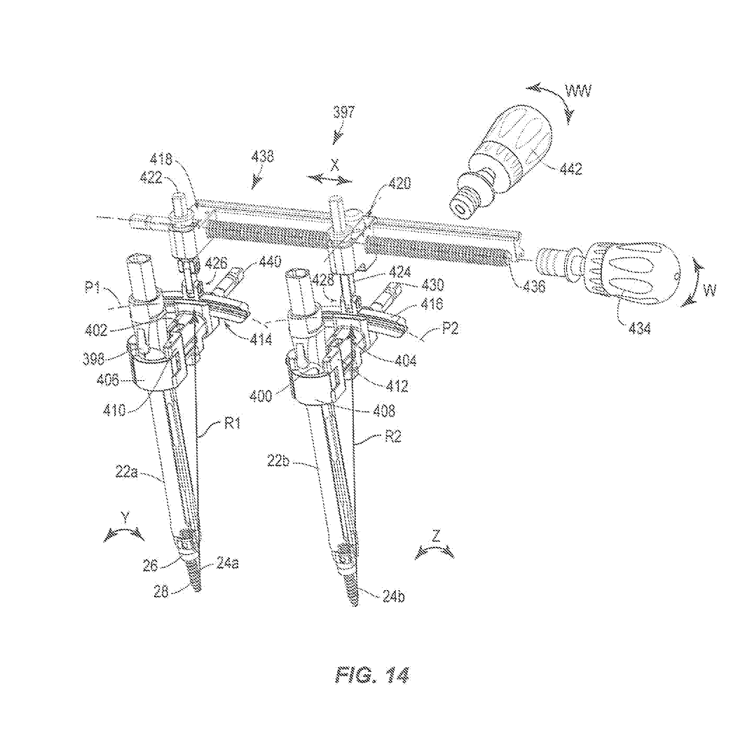

A surgical instrument 397, as shown in FIG. 14, comprises an alternate and interchangeable instrument that is matingly engaged with extenders 22a, 22b such that bone fasteners 24a, 24b are movable from a first configuration, which may include bone fasteners 24a, 24b being disposed in an initial axial position and in an initial angle relative to one another and a second configuration, which may include bone fasteners 24a, 24b being disposed at a corrected axial position and angle relative to one another.

Instrument 397 is disposed adjacent a surgical site and manipulated for engagement with bone fasteners 24a, 24b via extenders 22a, 22b. Extenders 22a, 22b are disposed in cavities 398, 400 of bodies 402, 404 and dips 406, 408 are engaged to ends 410, 412 of bodies 402, 404 to capture extenders 22a, 22b in cavities 398, 400, respectively.

Body 402 is disposed in a selected orientation relative to a gear rack 414, and body 404 is disposed in a selected orientation relative to a gear rack 416. Members 418, 420 are aligned with posts 422, 424, to connect members 418, 420 with coupling members 426, 428. A shaft 430 is oriented in the locking orientation such that translation of member 420 along shaft 430 does not cause relative movement between body 404 and gear rack 416 such that member 420, coupling member 428, body 404, extender 22b and bone fastener 24b axially translate as one unit.

A handle 434 is engaged to a shaft 436 of a rack 438 and rotated, in the directions shown by arrows W, such that member 420 with coupling member 428, body 404, extender 22b and bone fastener 24b axially translate along shaft 436, in the directions shown by arrows X, relative to rack 438, to apply a distraction or compression force to vertebrae. Member 420 axially translates relative to member 418 such that body 404 axially translates relative to body 402. Relative translation of bodies 402, 404 causes bone fasteners 24a, 24b, which are connected to bodies 402, 404 via extenders 22a, 22b, to substantially axially compress or distract the adjacent vertebra to restore vertebral body height of the vertebrae to treat the fractured vertebra.

Handle 442 is engageable and removable with bodies 402, 404 for connection to shafts 430 and/or 440 and actuating bodies 402, 404 for translation along their respective gear racks, as described herein. In one embodiment, shaft 440 is oriented in the first non-locking orientation so that body 402 translates along gear rack 414 along arcuate path P1 as shaft 440 is rotated by handle 442. Handle 442 is engageable with shaft 440 and rotated, in the directions shown by arrows WW, such that body 402 is translated along arcuate path P1 along gear rack 414 relative to member 418. Relative translation of body 402 along arcuate path P1 rotates bone fastener 24a, which is connected to body 402 via extender 22a, at a center of rotation from body 402 corresponding to radius R1. Bone fastener 24a rotates, as shown by arrows Y, to rotate vertebra to achieve lordosis and restore curvature of the vertebrae to treat the fractured vertebra. In some embodiments, handle 442 may be initially engaged with shaft 440 and subsequently engaged with shaft 430, as described herein. In some embodiments, handle 442 may be initially engaged with shaft 430 and subsequently engaged with shaft 440, as described herein. In some embodiments, handle 442 is engaged with shaft 430 only. In some embodiments, handle 442 is engaged with shaft 440 only.

In one embodiment, shaft 430 is oriented in the first non-locking orientation so that body 404 translates along gear rack 416 along arcuate path P2 as shaft 430 rotates. Handle 442 is removable and disengaged from shaft 440, and engaged with shaft 430. Handle 442 is rotated, in the directions shown by arrows WW, such that body 404 translates along arcuate path P2 along gear rack 416 relative to member 420. Relative translation of body 404 along arcuate path P2 rotates bone fastener 24b, which is connected to body 404 via extender 22b, at a center of rotation from body 404 corresponding to radius R2. Bone fastener 24b rotates, as shown by arrows Z, to rotate vertebra to achieve lordosis and restore curvature of the vertebrae to treat the fractured vertebra. In some embodiments, this configuration of instrument 397 compresses and/or distracts vertebrae to restore vertebral body height and restores curvature of the vertebrae by rotating vertebra about a center of rotation corresponding to a bone fastener adjacent a facet joint.

Upon completion of a procedure, as described herein, the surgical instruments, assemblies and non-implanted components of spinal implant system 20 are removed and the incision(s) are closed.

In some embodiments, instrument 397 is configured for engagement with spinal constructs to correct a spinal disorder, such as, for example, trauma and/or fracture of vertebrae, which may include a sagittal deformity, as described herein. In some embodiments, instrument 397 is employed to treat vertebrae such that selected vertebra can be relatively translated for substantially axial compression or distraction to restore vertebral body height and rotated to achieve lordosis and restore curvature of the spine.

In one embodiment, spinal implant system 20, similar to the systems and methods described herein with regard to FIGS. 1-14, is employed for treating a spine having an advanced deformity disorder and being sagittaly unbalanced such that the components of spinal implant system 20 engage spinal tissue along four or more vertebral levels.

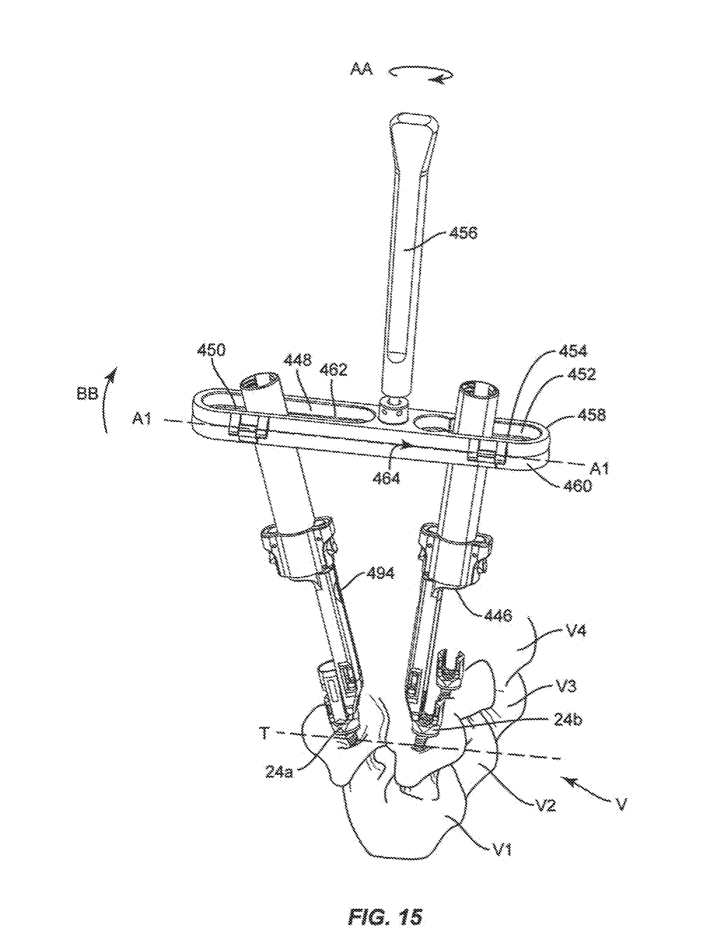

For example, spinal implant system 20 can be employed with a surgical correction treatment of an applicable condition or injury of an affected section of a spinal column and adjacent areas within a body, such as, for example, vertebral levels V1, V2, V3, V4 of vertebrae V, as shown in FIG. 15. A surgical pathway is created and pilot holes are made bilaterally in selected levels of vertebrae V, for example, vertebrae V1, V2 and V3 for receiving bone fasteners 24a, 24b. An extender 444 and an extender 446 are oriented for manipulation, alignment and capture of bone fasteners 24a, 24b. Extender 444 is configured for disposal with a concave portion of a selected vertebra of vertebrae V and extender 446 is configured for disposal with a convex portion of a selected vertebra of vertebrae V.

Extenders 444, 446 are attached with vertebra V1 via bone fasteners 24a, 24b, such that the components of spinal implant system 20 are disposed in a non-locking configuration. Extenders 444, 446 are matingly engaged with an alternate and interchangeable surgical instrument, such as, for example, a derotation handle, as described herein. Extenders 444, 446 are freely slidable for transverse translation within slots 448, 450 and/or slots 452, 454 of the derotation handle such that extenders 444, 446 are supported and can be selectively translated along slots 448, 450 and/or slots 452, 454 and positioned at a selected orientation.

A handle 456 is rotated in a clockwise direction, in the direction shown by arrow AA, such that a plate 458 is rotated relative to a plate 460 about an axis A1, in the direction shown by arrow BB, and driven apart from plate 460 to the locking configuration. Inner surfaces 462, 464 forcibly engage extenders 444, 446 in releasable fixation to fixedly dispose extenders 444, 446 in the selected position and/or orientation. Plates 458, 460 are locked with extenders 444, 446.

Force is applied to handle 456, for example, via manipulation of a practitioner during a surgical treatment, to displace, pull, twist or align vertebrae. Upon completion of practitioner manipulation, the components of spinal implant system 20 can be released from the locking configuration and pivoted to the non-locking configuration, as described.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for treating a spine, the method comprising the steps of:
providing a first surgical instrument comprising a surface having a mating element;
connecting the first surgical instrument with a fastener;
connecting the fastener with tissue;
engaging the mating element with a mating element of at least one of a plurality of alternate second surgical instruments interchangeable with the first surgical instrument; and
treating the spine with the first surgical instrument and the at least one second surgical instrument.

2. A method as recited in claim 1, wherein the first surgical instrument is configured such that the step of treating comprises one or more of engaging spinal tissue along one or two vertebral levels, engaging spinal tissue along three or four vertebral levels and engaging spinal tissue along four or more vertebral levels.

3. A method as recited in claim 1, wherein the first surgical instrument is configured such that the step of treating comprises engaging spinal tissue along three or four vertebral levels and the spine comprises a disorder that includes coronal unbalance and sagittal balance.

4. A method as recited in claim 1, wherein the first surgical instrument is configured such that the step of treating comprises engaging spinal tissue along four or more vertebral levels and the spine comprises a disorder that includes sagittal unbalance.

5. A method as recited in claim 1, wherein the first surgical instrument includes a first member and a second member that is axially translatable relative to the first member between an expandable configuration such that the second member engages the fastener and a non-expandable configuration such that the second member is locked with the fastener.

6. A method as recited in claim 1, further comprising the step of providing a lock movable to fix the first surgical instrument with the at least one second surgical instrument.

7. A method as recited in claim 6, wherein the lock includes a transverse member that is movable between a first position such that the transverse member is spaced from the at least one second surgical instrument and a second position such that the transverse member engages the at least one second surgical instrument.

8. A method as recited in claim 1, wherein the first surgical instrument includes a plurality of extenders and the step of engaging includes mating the extenders with a plurality of bodies of the at least one second surgical instrument, the at least one second surgical instrument being connectable with a spinal rod.

9. A method as recited in claim 8, wherein the bodies are disposed in a serial configuration.

10. A method as recited in claim 8, wherein the bodies are configured to space apart the extenders and each of the extenders are engageable with a fastener.

11. A method as recited in claim 8, wherein the at least one second surgical instrument includes a first member that includes the bodies and a second member connected to the spinal rod, the members being relatively movable to dispose the spinal rod with the extenders and in alignment with at least one of the fasteners.

12. A method as recited in claim 1, wherein the first surgical instrument includes a first extender and a second extender, and the step of engaging includes mating the first extender with a first member of the at least one second surgical instrument and mating the second extender with a second member of the at least one second surgical instrument such that the second member is axially translatable relative to the first member.

13. A method as recited in claim 12, wherein a first body is connected to the first member and is translatable along an arcuate path relative to the first member.

14. A method as recited in claim 13, wherein a second body is connected to the second member and is translatable along an arcuate path relative to the second member.

15. A method for treating a spine, the method comprising the steps of:
providing at least one extender comprising a surface having a mating element;
connecting the at least one extender with a bone screw;
connecting the bone screw with a vertebral level;
engaging the mating element with a mating element of at least one of a plurality of alternate second surgical instruments interchangeable with the at least one extender; and
treating the spine with the first surgical instrument, the at least one second surgical instrument and a complementary surgical instrument.

16. A method as recited in claim 15, wherein the first surgical instrument is configured such that the step of treating comprises one or more of engaging spinal tissue along one or two vertebral levels, engaging spinal tissue along three or four vertebral levels and engaging spinal tissue along four or more vertebral levels.

17. A method as recited in claim 15, wherein the complementary surgical instrument comprises a rod rotator engageable with a spinal rod disposed for alignment with the bone screw.

18. A method as recited in claim 15, wherein the complementary surgical instrument comprises a rod inserter engageable with a spinal rod disposed for alignment with the bone screw.

19. A method as recited in claim 15, wherein the complementary surgical instrument comprises a speculum having a mating element engageable with a spinal rod disposed for alignment with the bone screw.

20. A spinal implant system comprising:
a fastener;
a first surgical instrument comprising a surface having a mating element and a capture element engageable with the fastener; and
at least one of a plurality of alternate second surgical instruments, the at least one second surgical instrument having a mating element engageable with the mating element of the first surgical instrument such that the at least one second surgical instrument is interchangeable with the first surgical instrument in a configuration for use with one or a plurality of different surgical procedures for treating a spine.
